(19) United States
(12) Patent Application Publication
(10) Pub. No.: US 2020/0386587 A1
(43) Pub. Date: Dec. 10, 2020

(54) Environmental Monitoring Embodiment Comprising Multiple Analyzing, Assessment and Monitoring Components

(71) Applicant: David Gonzalez, Tonawanda, NH (US)

(72) Inventor: David Gonzalez, Tonawanda, NH (US)

(21) Appl. No.: 16/501,746

(22) Filed: Jun. 4, 2019

(51) Int. Cl. G01D 21/02; A61B 5/0205; A61B 5/1172

(52) U.S. Cl. CPC G01D 21/02; A61B 5/082; A61B 5/1172; A61B 5/02055

(57) ABSTRACT

This Embodiment revolutionizes the Medical and Environmental fields by combining a number of critical and life altering tools' to create a single comprehensive, easy to use, Laboratory. This Embodiment surpasses all existing Medical and Environmental tools by eliminating the dangers and restrictions of separation, size, and access. This Embodiment will provide, cheap, reliable, and dependable analysis in places where Doctors, Physicians, Scientist, and Professionals in the Medical and Environmental fields can and can't be. Today this Embodiment changes the way we operate, think, see, and live, introducing . . . Body Guard.

### BACKGROUND OF THE INVENTION

Environmental Monitoring has been around for decades, but limited by the separation of analyzing, assessment and monitoring components. CO2 Detectors exist in detecting CO2 admissions in a everyday product, and in a everyday changing environment, which is the case with all environmental monitoring devices. Environmental Monitoring biggest flaw is the separation portability and un-constant detection of environmental hazards. Vehicles sometimes contain toxic fumes, water sometimes contain dangerous particles, dirt sometimes contain harmful substances, temperatures sometimes can be dangerously low or high, all of which can be deadly without the proper Environmental Monitoring Devices. These problems which we endure in our everyday lives are very common, but not limited to, will be solved with the introduction of this invention.

### BRIEF SUMMARY OF INVENTION

This Embodiment combines a number of Environmental Monitoring software, and hardware, which would include but is not limited to drug testing, viral monitoring and detection, whether by (Blood/Urine/Saliva/Hair/Breathe). Said Embodiment will also be able to give negative and positive results for pregnancy or D.N.A. matches. Said Embodiment can also Monitor Blood Sugar and identify all listed and classified bacteria. Said Embodiment will contain a number of scanning hoppers inserts and analyzing ports. Said Embodiment is able to connect with all existing and future devices (Whether multi-media or not).

### DRAWINGS

FIG. 1 is a perspective outside front view of the embodiment

FIG. 2 is a perspective outside back view of the embodiment

FIG. 3 is a perspective outside bottom view of the embodiment

FIG. 4 is a perspective outside top view of the embodiment

FIG. 5 is a perspective outside right side view of the embodiment

FIG. 6 is a perspective outside left side view of the embodiment

### DETAIL DESCRIPTION

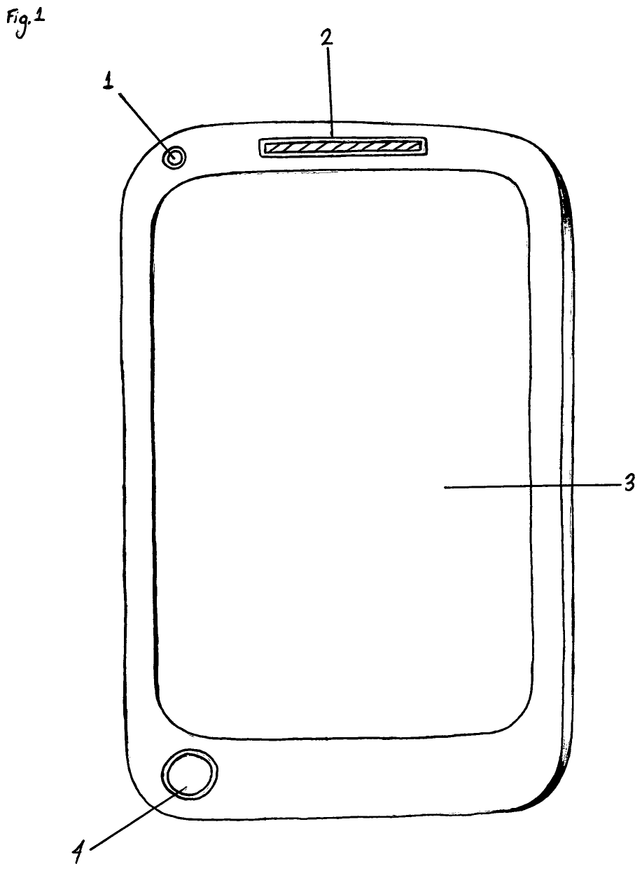

FIG. 1

Is a perspective view of the embodiments front side, showing Digital Thermometer,(1); Indoor and Outdoor Air Quality Meter,(2); Multiple LCD Screen (but not limited to),(3); Digital Breathalyzers/(but not limited to),(4);

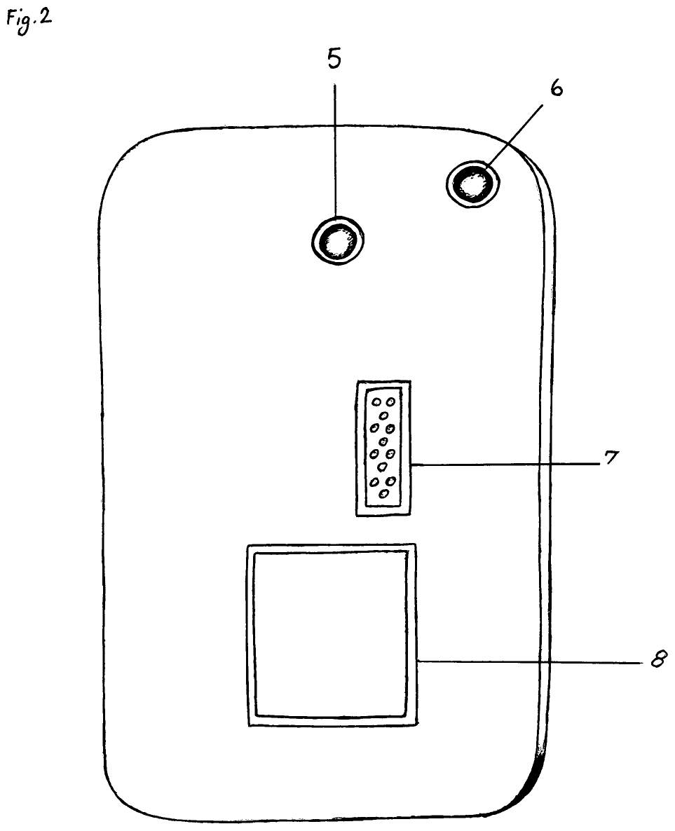

FIG. 2

Is a perspective view of the embodiments back side, showing High power/Low power Digital Microscope,(5); Digital Camera,(6); Digital Pulse Wave Analyzer,(7); Digital Fingerprint reader/scanner,(8);

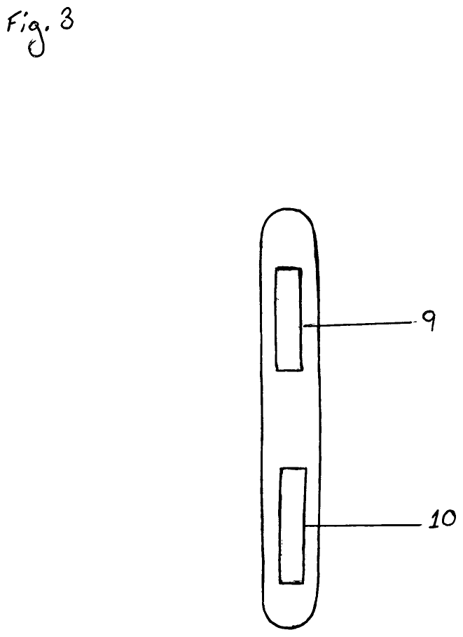

FIG. 3

Is a perspective view of the embodiments bottom side, showing Digital Food/Quality Tester (but not limited to),(9); Electrical Water Quality Tester (but not limited to),(10);

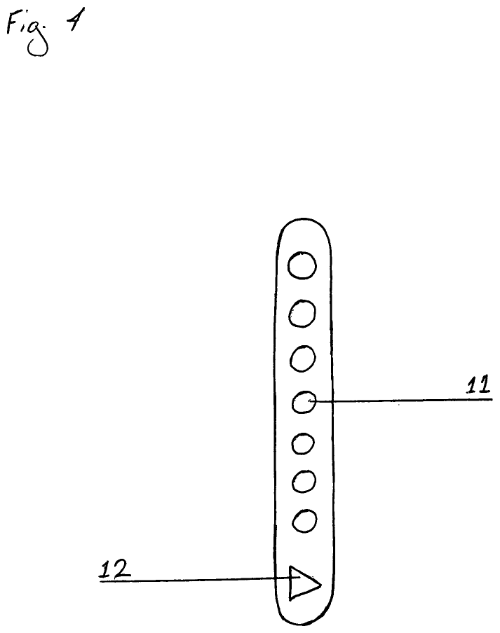

FIG. 4

Is a perspective view of the embodiments top side, showing a combination of UV light, blue light, green light, black light, white light, LED (but not limited to),(11); Ultra Violet Disinfection System,(12);

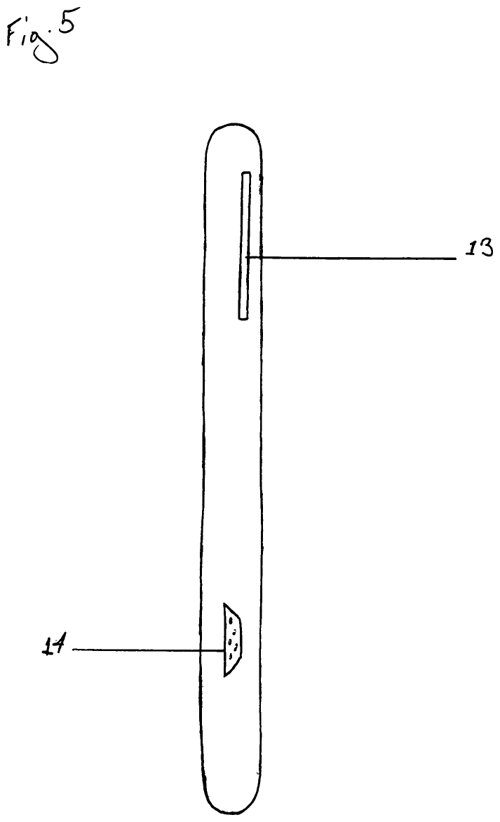

FIG. 5

Is a perspective view of the embodiments right side, showing memory expansion port,(13); Micro USB/Mini USB port,(14);

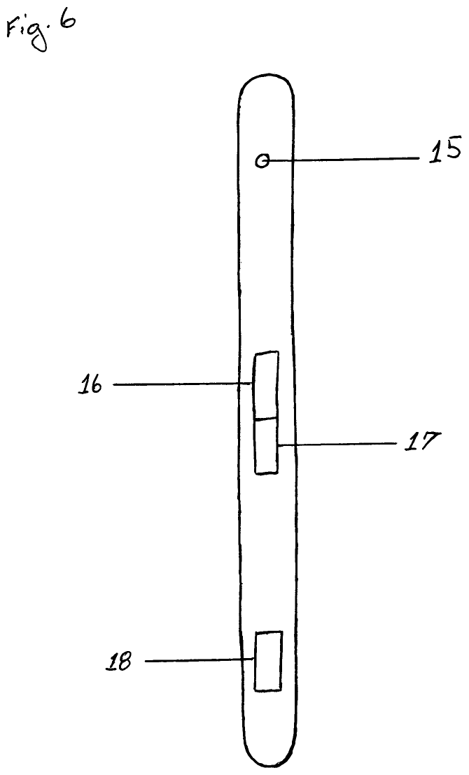

FIG. 6

Is a perspective view of the embodiments left side, showing non-contact infrared Laser Thermometer/reader,(15); Digital Pathogen and Micro Biomicrobiome Analyzer,(16); Digital Blood Analyzer/Tester,(17); Digital Urine Analyzer,(18).

### REFERENCE NUMBER(S)

(1) Digital Thermometer
(2) Indoor and Outdoor Air Quality Tester
(3) Multiple LCD (but not limited to) Screen
(4) Digital Breathalyzer/(but not limited to)
(5) High Power/Low Power Digital Microscope
(6) Digital Camera
(7) Digital Pulse Wave Analyzer
(8) Digital Fingerprint Reader/Scanner/On-Off-switch
(9) Digital Food/Quality Tester (but not limited to)
(10) Electrical Water Quality Tester (but not limited to)
(11) A combination of UV light, blue light, green light, black light, white light, LED (but not limited to)
(12) Ultra Violet Disinfection System
(13) Memory Expansion Port
(14) Micro USB/Mini USB Port
(15) Non-contact infrared Laser Thermometer/Reader
(16) Digital Pathogen and Micro Biomicrobiome Analyzer
(17) Digital Blood Analyzer/Tester
(18) Digital Urine Analyzer

### OPERATIONS

In operating said Invention, all hardware is controlled by way of software; Each component can be operated individually or simultaneously, some components operate automatically, for example Indoor and Outdoor Air Quality Meter is or can be set to auto-pilot, set to be manually turned on or activated and begin to pull in air (by way of suction)(but not limited to), which will then analyze air for any contaminants. (0001)

The Digital Thermometer can operate automatically or manually. When activated said component will give out digital readings of temperature by way of current thermometer technology and/or geological network based temperatures for outdoor temperature's, (but not limited to). (0002)

The Multiple LCD Screens can or will be color or black and/or be separated or single to provide best results based on components and technology at the time of building Invention. (0003)

The Digital Breathalyzer can be activated, manually or automatically. When activated, a sensor in the breathing hole, will detect a stream of breathe which in turn will enable software to being analyzing for alcohol or other substances. (0004)

The High or Low Power Microscope will use existing and/or future microscopic technologies to allow user to view on said LCD Screen whatever may be. Said Microscope is activated by way of software. (0005)

The Digital Camera is operated by way of optical software, it will use existing and/or future digital and component advancements in technologies. Once activated, said Camera can or will pair with Thermometer to analyze facial and/or temperamental differences compared to normal temperatures and/or pupil dilations (but not limited to). (0006)

The Digital Pulse Wave Analyzer will utilize pulse wave velocity to measure the status of the heart and blood vessels, arterial wall stiffness and biological age of arteries, (but not limited to). (0007)

The Digital Fingerprint/Scanner will use a digital image to capture fingerprints which in turn will pair with software to store the image automatically, to further be used in any biometrics applications; and serves as a On-Off switch for Invention. (0008)

The Digital Food/Quality Tester is a all in one food nitrate checker plus radiation detector, it also analysis gluten and pesticides. Said tester will operate by way of software to enable near-infrared spectrostopy to analyze all food, (but not limited to). (0009)

The Electrical Water Quality Tester will operate by way of software, to enable hardware, to do a total dissolved solids concentration test and enable paired photo meter to do an electronic water analysis for chlorine, Total Chlorine, Bromine, PH, Alkalinity, Calcium Hardness, Cyanuric acid, (but not limited to). (0010)

The combination of UV light, blue light, green light, black light, white light, LED, will or can be enabled by software or switch to emit what is known as a black light lamp that emits long wave ultra violet light to observe fluoresce organic substances and most bodily fluids including but not limited to sweat, saliva, urine, semen, blood. (0011)

The Ultra Violet Disinfection System can or will be extendible and activated by way of software or switch to provide a UV Lamp that provides rapid effective in activation of micro organisms through a physical process. (0012)

The Memory Expansion Port can and will be U.S.B., S.D., Micro S.D., U.S.B. plug but not limited to. (0013)

The Micro U.S.B./Mini U.S.B. Port can and will be U.S.B., S.D., Micro S.D., U.S.B. Plug but not limited to. (0014)

The non-contact Infrared Laser Thermometer is a infrared non-contact Thermometer which can or will be enabled by way of software to scan objects being measured that admits thermo-radiation. (0015)

The Digital Pathogen and Micro Biomicrobiome Analyzer can or will be enabled by way of software to analyze micro organism's in blood, fluid's or matter by way of (but not limited to) small sliding track, where samples can or will be placed and reinserted back into analyzer for analyzing. (0016)

The Digital Blood Analyzer/Tester can or will be activated by way of software to enable a glucose monitoring device, and or a non-invasive monitor which in turn can diagnose overall cardio vascular health, which would include but not limited to diseases, bacterial, blood sugar, blood pressure, D.N.A., pregnancy. (0017)

The Digital Urine Analyzer can or will be activated by way of software to enable a digital urinary microscopy to diagnose diseases, drug test, pregnancy test, but not limited to. (0018)

1. I claim a method of combining multiple Medical and Environmental monitoring software and hardware tools to create a embodiment capable but not limited to diagnosing, monitoring, enhancing, preventing, storing Medical and Environmental information.
